(19) United States
(12) Patent Application Publication
(10) Pub. No.: US 2022/0120749 A1
(43) Pub. Date: Apr. 21, 2022

(54) PROPHYLAXIS AND TREATMENT OF AN INFECTION OF THE CERVIX WITH HUMAN PAPILLOMAVIRUS (HPV)

(71) Applicant: Eberhard Karls Universität Tübingen Medizinische Fakultät, Tubingen (DE)

(72) Inventors: Matthias WILLMANN, Tubingen (DE); Thomas Iftner, Hirrlingen (DE)

(21) Appl. No.: 17/646,435

(22) Filed: Dec. 29, 2021

Related U.S. Application Data
(63) Continuation of application No. PCT/EP2020/068396, filed on Jun. 30, 2020.

(30) Foreign Application Priority Data
Jul. 2, 2019 (DE) 10 2019 117 837.1

(51) Int. Cl. G01N 33/574; A61K 31/7016; A61P 31/20

(52) U.S. Cl. CPC G01N 33/57411; A61P 31/20; A61K 31/7016

(57) ABSTRACT

A pharmaceutical composition is for prophylaxis and/or treatment of an infection of the cervix with human papillomavirus (HPV). A method carried out ex vivo is for predicting the probability of cure of HPV infection of the cervix. A biomarker is for predicting the emergence and/or development of cervical in-traepithelial neoplasia and/or cervical carcinoma and the likelihood of cure of cervical infection with HPV. Another method is for prophylaxis and/or treatment of cervical infection with HPV.

### CROSS-REFERENCES TO RELATED APPLICATIONS

This application is a continuation of copending International Patent Application PCT/EP2020/068396 filed on Jun. 30, 2020 and designating the United States, which was published under PCT Article 21(2) in a language other than English, and claims priority of German Patent Application DE 10 2019 117 837.1 filed on Jul. 2, 2019. The entire contents of these prior applications are incorporated herein by reference.

### FIELD

The present invention relates to a prophylaxis and/or treatment of human papillomavirus (HPV) infection of the cervix.

### BACKGROUND

Human papillomaviruses (HPV) are a group of DNA viruses that are now classified into more than 100 different types. HPV are non-enveloped, double-stranded DNA viruses (dsDNA) and belong to the family of Papillomaviridae and the genera Alpha papillomavirus, Beta papillomavirus and Gamma papillomavirus. They infect epithelial cells of the skin and various mucous membranes and can cause uncontrolled tumor-like growth in the infected cells.

However, some HPV types can also cause malignant changes. For example, persistent infection of the cervix with HPV for more than 12-24 months can lead to high-grade mucosal changes and cervical intraepithelial neoplasia (CIN) and ultimately to the development of cervical carcinoma in women.

There is currently no specific papillomavirus therapy.

In Germany, according to a new guideline of the Federal Joint Committee (Gemeinsamer Bundesausschuss, GbA), from 2020 women aged 35 and older will be offered cervical cancer screening, which consists of an HPV test and a cytological examination of the cervical mucosa. Women who are HPV positive with a high-risk virus (HR-HPV) and cytologically abnormal are further screened. Women who are HPV-negative and cytologically unremarkable will be recalled for the next screening examination in 3 years.

With regard to the further procedure for women who are HR-HPV positive but cytologically unremarkable, a new co-test is likely to be performed after 12 months. However, this strategy carries risks for the woman and for the cost-effectiveness of the screening. In most cases, HR-HPV infection heals spontaneously, so there is no increased risk of developing cervical cancer. However, these women have to wait 12 months for a negative result, which is an increased psychological burden. In the case of a persistent infection already present at the time of initial HPV testing, it is not possible to determine how long the persistent infection has been present or whether spontaneous healing may occur. In the case of a persistence of an HR-HPV infection for more than 24 months, there is an increased risk of developing a precancerous lesion or a CIN of intermediate (CIN II) or high grade (CIN III), which may develop into cervical carcinoma if undetected.

### SUMMARY

The present invention relates to a pharmaceutical composition for prophylaxis and/or treatment of human papillomavirus (HPV) infection of the cervix, a novel use related thereto, an ex vivo method for the prognosis of the probability of cure of infection of the cervix with HPV, a novel biomarker for predicting the emergence and/or development of cervical intraepithelial neoplasia and/or cervical carcinoma and the likelihood of cure of cervical infection with HPV, and a method for prophylaxis and/or treatment of cervical infection with HPV.

One aspect of the invention related to an ex vivo method for the prognosis of the probability of healing of an infection of the cervix with human papillomavirus (HPV), the method comprising providing a biological microbiome sample from a patient, examining the sample for the presence of Lactobacillus sp. with the metabolic pathway PWY-621, and prognosticating a high probability of cure in the presence of Lactobacillus sp. with the metabolic pathway PWY-621, or a low probability of cure in the absence of Lactobacillus sp. with the metabolic pathway PWY-621.

Another aspect of the invention is related to an ex vivo method for the prognosis of the emergence or development of a cervical intraepithelial neoplasia (CIN) or a cervical carcinoma, the method comprising providing a biological microbiome sample from a patient, examining the sample for the presence of Lactobacillus sp. with the metabolic pathway PWY-621, and prognosticating a low probability in the presence of Lactobacillus sp. with the metabolic pathway PWY-621, or an increased to high probability in the absence of Lactobacillus sp. with the metabolic pathway PWY-621.

### DETAILED DESCRIPTION

There is a need for early and prophylactic treatment of HPV infections, especially persistent HR-HPV infections, without pathological changes of the cervix. Therapeutic measures against a CIN of medium (CIN II) or high grade (CIN III) and an already developed cervical carcinoma are also necessary.

This object is achieved with the provision of a pharmaceutical composition for the prophylaxis and/or treatment of an infection of the cervix with human papillomavirus (HPV), which comprises at least one active ingredient, to promote the presence of Lactobacillus sp. with the metabolic pathway PWY-621 in the microbiome, as well as a pharmaceutically acceptable carrier.

The inventors have studied a large number of HPV-positive women. They have recognized that HPV-positive women in whom the infection heals spontaneously have a significantly higher number of lactobacilli characterized by the PWY-621 pathway compared to those in whom the infection persists.

The inventors were also able to realize that measures which promote the presence and cultivation of Lactobacillus sp. with the PWY-621 metabolic pathway in the microbiome can be used prophylactically and therapeutically in the case of infection of the cervix with HPV. Such measures could be, for example, the application of selective nutrients for the bacterium in question, e.g. via the vaginal mucosa.

The new pharmaceutical composition has few side effects, is inexpensive and easy to manufacture, even under GMP conditions.

These findings were surprising and not to be expected.

For example, it is known that vaginal administration of sucrose gel can alter the vaginal microbiota in rhesus macaques; see Hu et al. (2015), Directed shift of vaginal microbiota induced by vaginal application of sucrose gel in rhesus macaques, International Journal of Infectious Diseases 33, pp. 32-36. It has also been shown that vaginal application of sucrose can restore normal vaginal flora in patients affected by bacterial vaginosis. It has therefore been proposed to use vaginally applied sucrose gel for the treatment of bacterial vaginosis; Zeng et al. (2010), Directed shift of vaginal flora after topical application of sucrose gel in a phase III clinical trial: a novel treatment for bacterial vaginosis.

A prophylactic and/or therapeutic suitability of an active ingredient for viral infections, in particular those with HPV or HR-HPV, is not described in the prior art.

Lactobacillus sp. is a genus of gram-positive, mostly rod-shaped bacteria from the family Lactobacillaceae. Lactobacillus belongs to the lactic acid bacteria together with other bacterial genera. They all produce lactic acid by fermentation. Regarding their characteristics, the representatives of the genus Lactobacillus do not form a uniform group, therefore individual species are grouped into subgroups. Within the systematics it can be observed that individual species are newly added or no longer belong to the genus. This led to an extended description of the genus in 2009.

Lactobacillus sp. with the metabolic pathway PWY-621 is characterized by a metabolic pathway that is also referred to as “sucrose degradation III (sucrose invertase)” in the technical world. Through this metabolic pathway, the bacteria are able to degrade and utilize the disaccharide sucrose. Not all lactobacilli possess this ability. “PWY-621” is the so-called MetaCyc link, through which the metabolic pathway in question and organisms possessing this metabolic pathway can be found in the “MetaCyc” database (http://metacyc.ai.sri.com/).

“Promoting the presence” according to the invention means that the composition either causes a new cultivation of Lactobacillus sp. PWY-621 in the microbiome or increases the amount and/or concentration of Lactobacillus sp. PWY-621 already present there. In this regard, the inventors have found that the efficacy of prophylaxis and/or treatment of human papillomavirus (HPV) infection of the cervix improves with an increase in the concentration and/or amount of Lactobacillus sp. PWY-621.

According to the invention, the prophylaxis and/or treatment of human papillomavirus (HPV) infection of the cervix also comprises the prophylaxis and/or treatment of the emergence and/or development of a cervical intraepithelial neoplasia (CIN) and/or a cervical carcinoma.

Pharmaceutically acceptable carriers are sufficiently known in the prior art. They include, for example, binders, disintegrants, fillers, lubricants, as well as buffers, salts and other substances suitable for the formulation of drugs; see Rowe et al. (2012), Handbook of Pharmaceutical Excipients, 7^{th} Edition Pharmaceutical Press; or Bauer et al. (2017), Lehrbuch der pharmazeutischen Technologie, 10^{th} Edition, Wissenschaftliche Verlagsgesellschaft Stuttgart mbH. The contents of these publications are incorporated by reference into this application.

According to one embodiment of the pharmaceutical composition according to the invention, the HPVs are those of a high-risk type (HR-HPV).

This measure configures the pharmaceutical composition for those forms of HPV infection that are pathologically particularly relevant and pose a critical risk for affected women to develop intermediate-grade (CIN II) or high-grade (CIN III) CIN and ultimately cervical cancer disease.

High-risk HPVs include primarily HPV 16, 18, 31, and 33, but also 35, 39, 45, 51, 52, 56, 58, 59, 68, 73, and 82. In nearly every cervical cancer occurrence, at least one of the high-risk HPV groups is detectable in an HPV screen. HPV 26, 53, and 66 are also frequently counted among the high-risk HPV forms according to the invention.

In one embodiment of the invention, the pharmaceutical composition is configured for prophylaxis and/or treatment of a persistent infection of the cervix with HPV.

This measure provides, for the first time, a pharmaceutical composition that can be routinely used prophylactically in cases of positive HR-HPV and concomitant inconspicuous cervical mucosa. In the state of the art, such women are usually retested 12 months after the finding to determine whether spontaneous healing has occurred. However, prophylactic treatment is usually not given, although it cannot be determined how long a persistent infection has been present. However, if the HR-HPV infection persists for more than 24 months, there is an increased risk of developing a precancerous lesion (CIN II or CIN III, commonly CIN II+), which may develop into cervical carcinoma if undetected. This strategy means a high psychological burden and a considerable risk for the patient. The invention provides a remedy here, as prophylactic treatment can now be carried out in a low-risk manner after the initial diagnosis.

According to the invention, “persistent infection” or “persistence” means that the infection has been present for an extended period of time, namely at least 24 months (≥24 months).

According to one embodiment of the invention, the pharmaceutical composition according to the invention is adapted to promote the presence of Lactobacillus sp. having the metabolic pathway PWY-621 in the cervical and/or vaginal microbiome.

According to the invention, this measure has a targeted effect on the microbiome, which has a protective effect against HPV infections. The pharmaceutical composition according to the invention thus becomes particularly effective in that it promotes the self-healing powers of the organism.

According to one embodiment of the invention, the pharmaceutical composition has a formulation for intra-vaginal application.

With this measure, such an administration of the pharmaceutical composition according to the invention takes place, with which the development of the effectiveness is ensured at the location or in the vicinity of the origin of the disease.

In one embodiment of the invention, the formulation is one selected from the group consisting of: gel, cream, ointment.

This measure uses formulations that have proven particularly effective for an intra-vaginal application.

In a preferred embodiment of the invention, the at least one active ingredient is sucrose.

The inventors were able to determine that the application of sucrose leads to a selective enrichment of Lactobacillus sp. with the PWY-621 metabolic pathway in the patient's microbiome, making this agent particularly suitable for the invention.

Against this background, another subject matter of the present invention relates to the use of an agent for promoting the presence in the microbiome of Lactobacillus sp. having the metabolic pathway PWY-621, for the prophylaxis and/or treatment of human papillomavirus (HPV) infection of the cervix.

The features, properties, advantages and embodiments of the composition according to the invention apply equally to the use according to the invention.

Another subject matter of the invention relates to an ex vivo method for the prognosis of the probability of healing of an infection of the cervix with human papillomavirus (HPV), comprising the following steps:
Provision of a biological microbiome sample from a patient, preferably a cervical and/or vaginal swab,
Examination of the sample for the presence of Lactobacillus sp. with the metabolic pathway PWY-621, and
Prognosticating a
i) high probability of cure in the presence of Lactobacillus sp. with the metabolic pathway PWY-621, or
ii) low probability of cure in the absence of Lactobacillus sp. with the metabolic pathway PWY-621.

Another subject matter of the invention relates to ex vivo method for the prognosis of the emergence and/or development of a cervical intraepithelial neoplasia (CIN) and/or a cervical carcinoma, comprising the following steps:
Provision of a biological microbiome sample from a patient, preferably a cervical and/or vaginal swab,
Examination of the sample for the presence of Lactobacillus sp. with the metabolic pathway PWY-621, and
Prognosticating a
i) low probability in the presence of Lactobacillus sp. with the metabolic pathway PWY-621, or
ii) increased to high probability in the absence of Lactobacillus sp. with the metabolic pathway PWY-621.

The features, properties, advantages and embodiments of the composition according to the invention apply equally to the methods according to the invention.

Another subject matter relates to the use of Lactobacillus sp. with the PWY-621 metabolic pathway as a biomarker to predict the probability of cure of cervical infection with HPV.

The features, properties, advantages and embodiments of the composition according to the invention apply equally to the use according to the invention.

Another subject matter of the invention relates to a method for the prophylaxis and/or treatment of an infection of the cervix with HPV, comprising the application into a living being of at least one agent for promoting the presence in the microbiome of Lactobacillus sp. having the metabolic pathway PWY-621, wherein preferably the application is intra-vaginally, and further preferably the application is of the composition according to the invention and/or sucrose.

It is understood that the above features and those to be explained below can be used not only in the combination indicated in each case, but also in other combinations or on their own, without departing from the scope of the present invention.

The present invention will now be explained in more detail with reference to exemplary embodiments, from which further features, properties and advantages of the invention emerge. The exemplary embodiments are not restrictive.

It is also understood that individual features that are disclosed in the exemplary embodiments are disclosed not only in the context of the respective specific embodiment but in a generality and, taken by themselves, make their own contribution to the invention. The person skilled in the art can therefore freely combine these features with other features of the invention.

### Material and Methods

### Study Design

Case-control study design within a prospective observational study.

### Study Participants

100 women with positive evidence of human papillomavirus infection of the cervix uteri of a high-risk type (HR-HPV).

### Endpoint

Persistent infection of the HR-HPV infection after 24 months.

### Examined Exposure

Taxonomic and functional arrangement of the cervical microbiome at the initial examination.

### Methodology

Shotgun metagenomics on a HiSeq2500 with a 2×150 bp paired end approach and a sequencing depth of 4-5 Gb per sample, bioinformatics analysis with Trimmomatic/Kneaddata/Kaiju/Humann2, statistical regression models with Stata V13.

### Results

### HR-HPV Persistence

57 women had a transient infection which healed spontaneously within 24 months.

43 women had a persistent HR-HPV infection for at least 24 months.

### Microbiome Studies

The presence of lactobacilli with the marker PWY-621 proved to be a protective factor in logistic regression models (odds ratio=0.04, p=0.03). The value of this factor was confirmed in a Recursive Feature Elimination and Cross-Validated Selection. An increase in sucrose-degrading lactobacilli by the mean of the study population is expected to result in a 40.4% reduction in HR-HPV persistence risk. In conjunction with study results on intra-vaginal application of sucrose gel for the treatment of bacterial vaginosis and evidence that the same application in animal models could greatly increase the proportion of lactobacilli in the varginal/cervical microbiome, leads to the invention to use the same application for prophylaxis and/or therapy of persistent HR-HPV infections.

What is claimed, is:

1. A pharmaceutical composition for the prophylaxis and treatment of an infection of the cervix with human papillomavirus (HPV), comprising at least one active ingredient for promoting the presence in the microbiome of Lactobacillus sp. having the metabolic pathway PWY-621, and a pharmaceutically acceptable carrier.

2. The pharmaceutical composition according to claim 1, wherein said human papillomavirus (HPV) is of a high-risk type (HR-HPV).

3. The pharmaceutical composition according to claim 1, wherein said infection is a persistent infection of the cervix.

4. The pharmaceutical composition according to claim 1, wherein said microbiome is the cervical or vaginal microbiome.

5. The pharmaceutical composition according to claim 1, comprising a formulation for intra-vaginal application.

6. The pharmaceutical composition according to claim 5, wherein said formulation is selected from the group consisting of: gel, cream, and ointment.

7. The pharmaceutical composition according to claim 1, wherein the at least one active ingredient is sucrose.

8. A method for the prophylaxis or treatment of an infection of the cervix with human papillomavirus (HPV) in a human patient in need thereof, the method comprising:
administering to the patient the pharmaceutical composition according to claim 1.

9. The method according to claim 8, wherein said application is intra-vaginal.

10. A method for identification of a patient in need of prophylaxis or treatment of an infection of the cervix with human papillomavirus (HPV) and providing the prophylaxis or treatment to the patient, comprising:
providing a biological microbiome sample from the patient,
examining the sample for the presence or absence of Lactobacillus sp. with the metabolic pathway PWY-621,
identifying the absence of Lactobacillus sp. with the metabolic pathway PWY-621 in the sample, thereby identifying the patient as in need of the prophylaxis or treatment, and
providing the prophylaxis or treatment by increasing the concentration or amount of Lactobacillus sp. with the metabolic pathway PWY-621 in the patient.

11. The method according to claim 10, wherein the concentration or amount of Lactobacillus sp. with the metabolic pathway PWY-621 in the patient is increased by administering a pharamceutical composition to the patient, the pharmaceutical composition comprising at least one active ingredient for promoting the presence in the microbiome of Lactobacillus sp. having the metabolic pathway PWY-621, and a pharmaceutically acceptable carrier.

12. The method according to claim 10, wherein said human papillomavirus (HPV) is of a high-risk type (HR-HPV).

13. The method according to claim 10, wherein said infection is a persistent infection of the cervix.

14. The method according to claim 10, wherein said microbiome is the cervical or vaginal microbiome.

15. The method according to claim 11, wherein said pharamaceutical composition comprises a formulation for intra-vaginal application.

16. The method according to claim 15, wherein said formulation is selected from the group consisting of: gel, cream, and ointment.

17. The method according to claim 11, wherein the at least one active ingredient is sucrose.

18. The method according to claim 10, wherein the patient is in need of the prophylaxis or treatment due to a low probability of healing of an infection of the cervix with HPV.

19. The method according to claim 10, wherein the patient is in need of the prophylaxis or treatment due to a high probability of the emergence or development of a cervical intraepithelial neoplasia (CIN) or a cervical carcinoma.
